SEQUENCE LISTING (7 sequences)

SEQ ID NO: 1 - DNA, 42 bp, Artificial Sequence
  Mutagenic Oligonucleotide
ttacttggattctggaattcatttcggtgccaccaccacagc

SEQ ID NO: 2 - DNA, 23 bp, Artificial Sequence
  Sense PCR Primer
atattgacgggcagtatgcaatg

SEQ ID NO: 3 - DNA, 24 bp, Artificial Sequence
  Antisense PCR Primer
cctggtcctcatcgtttagcagtt

SEQ ID NO: 4 - DNA, 23 bp, Artificial Sequence
  Sense PCR Primer
catatggagaacactgcttaatg

SEQ ID NO: 5 - DNA, 22 bp, Artificial Sequence
  Antisense PCR Primer
ctcttctaagacactcctcttc

SEQ ID NO: 6 - DNA, 19 bp, Artificial Sequence
  Sense PCR Primer
tcaacccacttgtgctgtg

SEQ ID NO: 7 - DNA, 21 bp, Artificial Sequence
  Antisense PCR Primer
cgtggacaatggctactcaag

(19) United States
(12) Patent Application Publication
(10) Pub. No.: US 2006/0259989 A1
(43) Pub. Date: Nov. 16, 2006

(54) Sporadic tumor mouse model for drug discovery

(75) Inventors: Isabelle Coste-Invernizzi, Chazay d'Azergues (FR); Toufic Renno, Civrieux d'Azergues (FR)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/371,825

(22) Filed: Mar. 9, 2006

Related U.S. Application Data
(60) Provisional application No. 60/660,956, filed on Mar. 11, 2005.

(51) Int. Cl. A01K 67/027; C12N 5/06

(52) U.S. Cl. 800/10; 800/18; 435/354

(57) ABSTRACT

The present invention relates to a transgenic mouse containing a latent oncogenic β-catenin allele capable of spontaneous activation in vivo. The transgenic mouse is useful for the identification of compounds and immunotherapies for the prevention, treatment and/or cure of various forms of cancer associated with β-catenin activation.

### BACKGROUND OF THE INVENTION

Beta-catenin is a key member of the highly-conserved, developmentally regulated Wnt signal transduction pathway that has been shown to play a critical role in embryonic development and oncogenic transformation (Polakis, Genes & Dev. 14:1837 (2000)). Beta-catenin plays a structural role in the assembly of cell-cell adherens junctions by forming a complex with cadherin adhesion receptors, α-catenin, and actin (Conacci-Sorrell et al., J. Clin. Invest. 109:987 (2002)). Perhaps more importantly, β-catenin can act as a transcription factor in the nucleus by serving as a coactivator of the lymphoid enhancer factor (LEF)/TCF family of DNA-binding proteins (Id.).

The level of β-catenin is controlled through Wnt/wingless signaling. In the absence of Wnt signaling, a multiprotein complex including adenomatous polyposis coli (APC), axin, β-catenin and glycogen synthase kinase-3β (GSK3β) induces phosphorylation of serine and threonine residues in the amino terminus of β-catenin, which targets β-catenin for ubiquitinylation and subsequent degradation by the proteosome (Polakis, Genes & Dev. 14:1837 (2000)). Wnt/wingless signaling activates frizzled receptors and, through disheveled, inhibits the ability of this multiprotein complex to phosphorylate β-catenin, leading to an increase in cytoplasmic β-catenin (Id.). As a result, β-catenin accumulates in the nucleus and activates LEF/TCF target genes, such as cyclin D1, Myc, fibronectin, matrilysin, and Mdr 1 (Conacci-Sorrell et al., J. Clin. Invest. 109:987 (2002)).

Mutations in the amino-terminal serine and threonine residues of β-catenin, and amino acids adjacent to them, inhibit the phosphorylation and subsequent degradation of the protein (Morin et al., Science 275:1787 (1997); Rubinfeld et al., Science 275:1790 (1997)). Such stabilizing mutations have been identified in a wide variety of human cancers, such as colon cancer (Sparks et al., Cancer Res. 58:1130 (1998)), brain cancer (Zurawel et al., Cancer Res. 58:896 (1998)), gastric cancer (Park et al., Cancer Res. 59:4257 (1999)), thyroid cancer (Garcia-Rostan et al., Cancer Res. 59:1811 (1999)), skin cancer (Rimm et al., Am. J. Pathol. 154:325 (1999)), prostate cancer (Voeller et al., Cancer Res. 58:2520 (1998)), kidney cancer (Koesters et al., Cancer Res. 59:3880 (1999)), liver cancer (de La Coste et al., Proc. Natl. Acad. Sci. USA 95:8847 (1998)), ovarian cancer (Palacios and Gamallo, Cancer Res. 58:1344 (1998)), and uterine cancer (Fukuchi et al., Cancer Res. 58:3526 (1998)).

In an effort to create models of these human cancers, several transgenic mouse strains have been developed which overexpress β-catenin in selected tissues. These “gain-of-function” mice exhibit a range of phenotypes, including hyperplasia and hepatomegaly of the liver (Harada et al., Cancer Res. 62:1971 (2002); Cadoret et al., Cancer Res. 61:3245 (2001)), dysplasia and polyposis of the intestine (Harada et al., EMBO J. 18:5931 (1999); Romagnolo et al., Cancer Res. 59:3875 (1999)), and mesenchymal cell, hair follicle, and mammary gland carcinoma (Cheon et al., Proc. Natl. Acad. Sci. USA 99:6973 (2002); Michaelson and Leder, Oncogene 20:5093 (2001); Gat et al., Cell 95:605 (1998)).

However, the use of these mouse strains as models of human cancers suffers from several drawbacks. First, the promoters used to drive expression of the transgene do so too early in development, leading to possible immunological tolerance. Second, such strains often contain non-physiological levels of transgene due to multiple integration of the transgene. Third, unlike human tumors, which generally clonally arise from one mutant cell, a group or “field” of many transformed cells arises when a promoter is activated in a transgenic mouse.

As a result, there is an immediate need for a transgenic mouse comprising a latent, oncogenic allele of β-catenin capable of spontaneous activation in vivo. Such a transgenic mouse strain would be useful as a model for identification of anti-cancer molecules and therapies in a setting that most closely mimics the mechanisms involved in human cancers.

### SUMMARY OF THE INVENTION

The present invention fills the foregoing need by providing a transgenic β-catenin mouse whose genome comprises a latent oncogenic β-catenin allele capable of spontaneous activation in vivo. The transgenic mouse is useful in the identification of reagents for the prevention and treatment of various forms of cancer associated with β-catenin activation.

The present invention also provides a transgenic β-catenin mouse comprising at least one cell expressing an oncogenic form of β-catenin, wherein the genome of the transgenic mouse comprises a latent oncogenic β-catenin transgene which upon spontaneous activation in vivo results in at least one cell of the transgenic mouse expressing an oncogenic form of β-catenin, and wherein expression of the oncogenic form of β-catenin results in the transgenic mouse developing a clonally derived sporadic tumor.

The present invention further provides a method of producing a transgenic β-catenin mouse comprising a latent oncogenic allele of β-catenin capable of spontaneous activation in vivo comprising: (a) providing a transgene comprising an oncogenic β-catenin nucleotide sequence; (b) introducing the transgene into mouse embryonic stem cells; (c) selecting embryonic stem cells that have integrated the transgene by homologous recombination such that a latent oncogenic β-catenin allele is formed; (d) introducing the embryonic stem cells containing the transgene into mouse blastocysts; (e) transplanting the blastocytes into a pseudopregnant mouse; (f) allowing the embryo to develop to term, producing a chimeric founder transgenic mouse; and (g) breeding chimeric transgenic mice to obtain F1 mice heterozygous for the transgene.

The present invention further provides a method for identifying a compound for treating a cancer associated with β-catenin activation comprising administering to a first β-catenin transgenic mouse a candidate agent and determining a beneficial effect of the candidate agent upon the first transgenic mouse as compared to a second β-catenin transgenic mouse not administered the agent.

The present invention further provides a double transgenic mouse whose genome comprises a latent oncogenic allele of β-catenin capable of spontaneous activation in vivo and a reporter transgene operably linked to a β-catenin-inducible promoter. Such double transgenic mice can be used to identify compounds for treating a cancer associated with β-catenin activation by monitoring changes in reporter activity.

The present invention further provides a double transgenic mouse whose genome comprises a latent oncogenic allele of β-catenin capable of spontaneous activation in vivo and a HLA-A24 transgene. Such double transgenic mice can be used to identify antigen-specific cancer immunotherapies for treating a cancer associated with β-catenin activation.

### DETAILED DESCRIPTION

The present invention provides a transgenic mouse comprising a latent oncogenic allele of β-catenin capable of spontaneous activation in vivo. Upon activation, the transgenic mouse expresses an oncogenic form of β-catenin in at least one of its cells, resulting in the development of a clonally derived sporadic tumor. Such tumor development more closely mimics the mechanisms involved in human cancers than those seen in traditional transgenic animals.

As used herein, a “transgenic mouse” refers to a mouse that contains a “transgene” integrated into the genetic material of at least one of its cells. “Transgene” refers to an exogenous nucleic acid molecule that is placed into an organism by introducing the exogenous nucleic acid molecule into embryonic stem (ES) cells, newly fertilized eggs or early embryos. The term “exogenous nucleic acid molecule” refers to any nucleic acid which is introduced into the genome of a cell by experimental manipulations and may include nucleic acid sequences found in that animal so long as the introduced nucleic acid sequence contains some modification (e.g., a point mutation, the presence of a selectable marker gene, the presence of a loxP site, etc.) relative to the endogenous nucleic acid sequence.

The term “gene” refers to a DNA sequence that comprises control and coding sequences necessary for the production of a polypeptide or its precursor. The polypeptide can be encoded by a full length coding sequence (either genomic DNA or cDNA) or by any portion of the coding sequence so long as the desired activity is retained.

The term “wild-type” refers to a nucleic acid or protein that has the characteristics of that nucleic acid or protein when isolated from a naturally occurring source. A wild-type nucleic acid or protein is that which is most frequently observed in a population and is thus arbitrarily designated the “normal” or “wild-type” form of that molecule. In contrast, the term “modified” or “mutant” refers to a nucleic acid or protein which displays modifications in sequence and or functional properties (i.e., altered characteristics) when compared to the wild-type nucleic acid or protein.

As used herein, the terms “non-transgenic mouse” and “normal mouse” refer to a wild-type mouse that does not contain a transgene.

The terms “in operable combination”, “in operable order” or “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The terms “promoter element” or “promoter” refer to a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences.

The terms “selectable marker” or “selectable gene product” refer to the use of a gene which encodes an enzymatic activity that confers resistance to an antibiotic or drug upon the cell in which the selectable marker is expressed. Selectable markers may be “positive”; positive selectable markers typically are dominant selectable markers, i.e., genes which encode an enzymatic activity which can be detected in any mammalian cell or cell line (including ES cells). Examples of dominant selectable markers include the bacterial aminoglycoside 3′ phosphotransferase gene (also referred to as the neo gene) which confers resistance to the drug G418 in mammalian cells, the bacterial hygromycin G phosphotransferase (hyg) gene which confers resistance to the antibiotic hygromycin, and the bacterial xanthine-guanine phosphoribosyl transferase gene (also referred to as the gpt gene) which confers the ability to grow in the presence of mycophenolic acid. Selectable markers may also be “negative”; negative selectable markers encode an enzymatic activity whose expression is cytotoxic to the cell when grown in an appropriate selective medium. For example, the HSV thymidine kinase (tk) gene is commonly used as a negative selectable marker. Expression of the tk gene in cells grown in the presence of gancyclovir or acyclovir is cytotoxic; thus, growth of cells in selective medium containing gancyclovir or acyclovir selects against cells capable of expressing a functional tk enzyme.

The terms “targeting vector” or “targeting construct” refer to nucleotide sequences comprising a selectable marker flanked on either side by β-catenin nucleotide sequences. The targeting vector contains β-catenin nucleotide sequences sufficient to permit the homologous recombination of the targeting vector into at least one allele of the endogenous β-catenin gene in the chromosomes of the target or recipient (e.g., ES) cells. The targeting vector may contain more than one selectable maker gene. When more than one selectable marker gene is employed, the targeting vector preferably contains a positive selectable marker (e.g., neo) and a negative selectable marker (e.g., tk). The presence of the positive selectable marker permits the selection of recipient cells containing an integrated copy of the targeting vector whether this integration occurred at the target site or at a random site. The presence of the negative selectable marker permits the identification of recipient cells containing the targeting vector at the targeted site (i.e., which has integrated by virtue of homologous recombination into the target site).

The term “tumor” refers to an abnormal mass or population of cells that result from excessive cell division, whether malignant or benign, and all pre-cancerous and cancerous cells and tissues. A “tumor” is further defined as two or more neoplastic cells. A “sporadic” tumor as used herein is a tumor that arises from in vivo activation of a latent oncogenic β-catenin allele. A “clonally derived” sporadic tumor as used herein is a sporadic tumor that develops from a single cell that begins to proliferate abnormally as a result of in vivo activation of a latent oncogenic β-catenin allele. The clonal origin of a tumor does not, however, imply that the original progenitor cell that gives rise to a tumor has initially acquired all of the characteristics of a cancer cell. Such characteristics can arise any time after tumor initiation.

The terms “treating” or “treatment” of a disease refer to executing a protocol, which may include administering one or more drugs to a patient (human or otherwise), in an effort to alleviate signs or symptoms of the disease. Thus, “treating” or “treatment” does not require complete alleviation of signs or symptoms, does not require a cure, and specifically include protocols that have only marginal effect on the patient.

The methods of the present invention utilize routine techniques in the field of molecular biology. Basic texts disclosing general molecular biology methods include Sambrook et al., Molecular Cloning, A Laboratory Manual (3d ed. 2001), Ausubel et al., Current Protocols in Molecular Biology (1994), and Hogan et al., Manipulating the Mouse Embryo, A Laboratory Manual (1994).

The β-catenin transgenic mouse of the present invention is preferentially generated by introduction of a targeting vector into murine ES cells. ES cells are grown on an appropriate fibroblast-feeder layer or grown in the presence of appropriate growth factors, such as leukemia inhibiting factor (LIF). Targeting vectors containing transgenes can be efficiently introduced into the ES cells by DNA transfection using a variety of methods known to the art, including electroporation, calcium phosphate co-precipitation, protoplast or spheroplast fusion, lipofection and DEAE-dextran-mediated transfection. Transgenes may also be introduced into ES cells by retrovirus-mediated transduction or by micro-injection. Cells containing the targeting vector may be detected by employing a selective medium. After sufficient time for colonies to grow, they are picked and analyzed for the occurrence of homologous recombination or integration of the construct using, e.g., Southern blot or PCR. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for cells that have incorporated the targeting vector. By providing for a different phenotype of the blastocyst and the ES cells, chimeric progeny can be readily detected. The chimeric animals are screened for the presence of the transgene. Chimeric animals containing the transgene (normally chimeric males) are mated with wildtype animals to produce heterozygotes, and the heterozygotes are mated to produce homozygotes if desired.

The targeting vector for producing the β-catenin transgenic mouse of the present invention will contain at least a portion of a β-catenin gene containing a modification or mutation capable of initiating oncogenesis associated with β-catenin activation (i.e., oncogenic form of β-catenin). Nucleic acid sequences encoding β-catenin from various species are publicly available from Genbank and include human (Acc. No. NM_{—}001904), mouse (Acc. No. NM_{—}007614), rat (Acc. No. NM_{—}053357) chicken (Acc. No. NM_{—}205081), pig (Acc. No. NM_{—}214367), zebrafish (Acc. No. NM_{—}001001889), frog (Acc. No. BC082826), and sea squirt (Acc. No. AB031543). Preferably, the β-catenin gene is of murine origin.

Modifications and mutations in the β-catenin gene include substitutions, insertions, deletions, rearrangements, and the like and can be introduced using methods well known in the art, such as site-directed mutagenesis using PCR. Preferably, the mutation alters an amino-terminal serine or threonine residue of β-catenin, or an amino acid adjacent to them, such that post-translational phosphorylation and subsequent degradation is inhibited (see Polakis, Genes & Dev. 14:1837 (2000)). More preferably, the mutation replaces the serine at residue 37 in exon 3 of β-catenin with a phenylalanine (S37F).

The targeting vector is preferably an insertion type vector such that homologous recombination at the endogenous murine β-catenin locus results in positive ES clones having a wild-type β-catenin allele on one chromosome and a latent (i.e., non-functional) oncogenic β-catenin allele made up of wild-type sequence and mutant or modified sequence separated by plasmid and neomycin-resistant sequences on the other chromosome (see Hasty et al., Nature 350:243 (1991); Valancius and Smithies, Mol. Cell. Biol. 11:1402 (1991)). Such insertion type targeting vectors carrying mutant alleles are routinely used to generate mice carrying subtle mutations in endogenous genes (Bradley et al., Int. J. Dev. Biol. 42:943 1998)). However, instead of selecting for resolution of the duplication in vitro (using a negative selection marker such as tk), as is done with the “hit-and-run” technique of Hasty et al. and Valancius and Smithies, applicants have found that introduction of the targeted ES cells into a blastocyst without in vitro selection results in transgenic animals whose dividing cells will resolve the duplication in the latent oncogenic β-catenin allele by an intrachromosomal or sister chromatid recombination event at a low frequency in vivo. One half the cells undergoing a recombination event in vivo are expected to generate a dominant, oncogenic β-catenin allele (i.e., spontaneous activation in vivo), and the other half a wild-type β-catenin allele.

As described in detail below, transgenic mice containing an oncogenic β-catenin allele display lesions associated with various types of neoplasia upon spontaneous activation in vivo, including peri-mammary skin papillomas and intestinal-type gastric metaplasia and dysplasia (i.e., clonally derived sporadic tumors). As such, the β-catenin transgenic mouse of the present invention provides an animal model for human cancer and provides a screening method for identifying compounds having anti-cancer activity in a setting that most closely mimics the mechanisms involved in human cancers. In general, the method involves administering to a first β-catenin transgenic mouse a candidate agent and determining the beneficial effect of the candidate agent upon the first β-catenin transgenic mouse as compared to a second β-catenin transgenic mouse not administered the agent. The candidate agent can be administered to the β-catenin transgenic mouse prior to or following spontaneous activation of the latent oncogenic β-catenin allele (e.g., prior to or following tumor formation).

Agents can be screened for their ability to affect β-catenin function (e.g., reduced activation of LEF/TCF target genes) or to mitigate an undesirable phenotype (e.g., tumor formation) associated with β-catenin expression. The indices used preferably are those which can be detected in a live animal, such as development or progression of tumors. Delayed death, or prevention, reduction, or regression of tumor formation (or some other phenotype associated with β-catenin expression), is an indication of the effectiveness of the candidate agent. The effectiveness can be confirmed by effects on pathological changes when the animal dies or is sacrificed. Such agents are candidates for development of treatments for cancers associated with β-catenin activation, such as colon cancer, brain cancer, gastric cancer, thyroid cancer, skin cancer, prostate cancer, kidney cancer, liver cancer, ovarian cancer, and uterine cancer.

The term “agent” or “compound” as used herein describes any molecule, e.g. protein or pharmaceutical, with the capability of altering the oncogenic potential of β-catenin. For example, an agent may be identified using the screening methods of the invention. Generally, a plurality of assays are run in parallel with different agent concentrations to obtain a differential response to the various concentrations. Typically, one of these concentrations serves as a negative control, i.e., at zero concentration or below the level of detection. Although the method generally is used as a screening assay to identify previously unknown molecules that can act as a therapeutic agent, the method can also be used to confirm and standardize the desired activity of a therapeutic agent.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to, peptides, saccharides, fatty acids, steroids, purines, pyrimidines, and various derivatives, structural analogs and combinations thereof.

Candidate agents can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

The route of administration of a candidate agent will depend, in part, on the chemical structure of the candidate agent. Peptides and polynucleotides, for example, are not particularly useful when administered orally because they can be degraded in the digestive tract. However, methods for chemically modifying peptides, for example, to render them less susceptible to degradation by endogenous proteases or more absorbable through the alimentary tract are well known. In addition, a peptide agent can be prepared using D-amino acids, or can contain one or more domains based on peptidomimetics, which are organic molecules that mimic the structure of peptide domain, or can be based on a peptoid such as a vinylogous peptoid.

A candidate agent can be administered to a subject by various routes including, for example, orally or parenterally, such as intravenously, intramuscularly, subcutaneously, intraorbitally, intratumorally, intracapsularly, intraperitoneally, intrarectally, intracisternally, or by passive or facilitated absorption through the skin using, for example, a skin patch or transdermal iontophoresis, respectively. Furthermore, the candidate agent can be administered by injection, intubation, or orally or topically, the latter of which can be passive by, for example, direct application of an ointment, or active using, for example, a nasal spray or inhalant, in which case one component of the composition is an appropriate propellant.

The total amount of a candidate agent can be administered to a subject as a single dose, either as a bolus or by infusion over a relatively short period of time, or can be administered using a fractionated treatment protocol, in which multiple doses are administered over a prolonged period of time. The candidate agent can be formulated for oral formulation, such as a tablet, or a solution or suspension form, or can comprise an admixture with an organic or inorganic carrier or excipient suitable for enteral or parenteral applications. The candidate agent can be compounded with the usual non-toxic, pharmaceutically acceptable carriers for tablets, pellets, capsules, suppositories, solutions, emulsions, suspensions, or other form suitable for use. Such carriers include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form.

Another screening method involves the crossing of the transgenic β-catenin mouse of the present invention with mice harboring reporter transgenes under the control of a β-catenin-inducible promoter, such as LEF/TCF (a transgenic LEF/TCF-reporter mouse). The double transgenic offspring can be used to identify compounds having anti-cancer activity by monitoring changes in reporter activity.

Methods for making a transgenic reporter mouse are well known in the art. Once such method is zygote injection (see, e.g., U.S. Pat. No. 4,873,191), which involves injecting the reporter transgene into a zygote (general the larger male pronucleus) and allowing the egg to develop in a pseudo-pregnant mother. The zygote can be obtained using male and female animals of the same strain or from male and female animals of different strains. The animals born are screened for the presence of the desired integrated transgene by, e.g., Southern blot or PCR. A resulting transgenic mouse is called a founder, and it is bred to produce more animals with the same transgene insertion. The use of zygotes as a target for gene transfer has a major advantage in that in most cases one to many thousands of copies of the transgene will be incorporated into the host genome before the first cleavage. As a consequence, all cells of the transgenic mouse will carry the incorporated one or more copies of the transgene. This will in general also be reflected in the efficient transmission of the transgene to offspring of the founder since 50% of the germ cells will harbor the transgene.

Alternatively, a transgenic reporter mouse can be produced by introduction of the transgene into ES cells as described above. The advantage of this method is that the ES cells can be screened for appropriate copy number and integration arrangement prior to blastocyst injection.

Retroviral transfection can also be used to produce a transgenic reporter mouse. The developing mouse embryo can be cultured in vitro to the blastocyst stage. During this time, the blastomeres can be targets for retroviral infection. Efficient infection of the blastomeres is obtained by enzymatic treatment to remove the zona pellucida. The viral vector system used to introduce the reporter transgene is typically a replication-defective retrovirus carrying the transgene. Transfection is easily and efficiently obtained by culturing the blastomeres on a monolayer of virus-producing cells. Alternatively, infection can be performed at a later stage. Virus or virus-producing cells can be injected into the blastocoele. Most of the founders will be mosaic for the reporter transgene since incorporation occurs only in a subset of cells which form the transgenic animal. Further, the founder may contain various retroviral insertions of the reporter transgene at different positions in the genome which generally will segregate in the offspring. In addition, it is also possible to introduce reporter transgenes into the germline, albeit with low efficiency, by intrauterine retroviral infection of the midgestation embryo.

Reporter molecules, which confer a detectable phenotype on a cell, are well known in the art and include, for example, fluorescent polypeptides such as green fluorescent protein, cyan fluorescent protein, red fluorescent protein, or enhanced forms thereof; an antibiotic resistance polypeptide such as puromycin N-acetyltransferase, hygromycin B phosphotransferase, aminoglycoside phosphotransferase, and the Sh ble gene product; a cell surface protein marker such as the cell surface protein marker neural cell adhesion molecule (N-CAM); an enzyme such as β-lactamase, chloramphenicol acetyltransferase, adenosine deaminase, aminoglycoside phosphotransferase, dihydrofolate reductase, thymidine kinase, luciferase or xanthine guanine phosphoribosyltransferase polypeptide; or a tag such as a c-myc peptide, a polyhistidine, a FLAG epitope, or any ligand (or cognate receptor), including any peptide epitope (or antibody, or antigen binding fragment thereof, that specifically binds the epitope). Expression of a reporter molecule can be detected using the appropriate instrumentation or reagent, for example, by detecting fluorescence of a green fluorescent protein or light emission upon addition of luciferin to a luciferase reporter molecule, or by detecting binding of nickel ion to a polypeptide containing a polyhistidine tag. Similarly, expression of a selectable marker such as an antibiotic can be detected by identifying the presence of cells growing under the selective conditions. Preferably, the reporter can be detected without sacrificing the mouse.

For example, a transgenic mouse can be produced in which each cell of its genome contains a luciferase transgene under the control of the LEF/TCF promoter. The transgenic reporter mouse is bred to a mouse containing a latent oncogenic β-catenin allele, and the resulting offspring screened for the presence of both the reporter transgene and oncogenic allele. Luciferase expression will be induced in those cells of the double transgenic mouse in which spontaneous activation of the latent oncogenic β-catenin allele occurs. Such animals provide a non-invasive, sensitive screening method for identifying compounds having anti-cancer activity. Candidate agents can be screened for their ability to reduce luciferase expression, without the need to sacrifice the animal. Changes in luciferase expression can be monitored in living animals using, e.g., a charge-coupled device (CCD) camera following administration of a luciferin substrate. A mouse can be placed in a light-tight dark box and projection images of CCD-imaged bioluminescence can be superimposed on photographic images of the mouse to image quantitatively and repetitively the bioluminescent signal from a given location (De et al., Mol. Ther. 7:681 (2003); Massoud and Gambhir, Genes Dev. 17:545 (2003); Honigman et al., Mol. Ther. 3:239 (2001); Contag et al., Neoplasia 2:41 (2000)). Reduced bioluminescence at a given location following administration of a candidate agent, as compared to a double transgenic mouse not administered a candidate agent (or a double transgenic animal prior to administration), is an indication of the effectiveness of the candidate agent.

The transgenic β-catenin mouse of the present invention also finds utility as an animal model for cancer immunotherapy development. The mutant S37F form of β-catenin has been shown to be a melanoma-specific antigen recognized by HLA-A24 restricted tumor-infiltrating lymphocytes (TILs) (Robbins et al., J. Exp. Med. 183:1185 (1996)). As such, a transgenic mouse expressing a latent oncogenic S37F β-catenin allele that undergoes spontaneous activation in vivo, as well as HLA-A24, would provide a setting that closely mimics that seen in human cancers for developing cancer vaccines. In this respect, a transgenic β-catenin mouse of the present invention can be crossed with a transgenic HLA-A24 mouse, and the resulting offspring screened for the presence of both the HLA-A24 transgene and oncogenic allele. Preferably, the transgenic HLA-A24 mouse is a transgenic HLA-A2402/K^{b} mouse. Such a mouse, expressing the human α1 and α2 domains of HLA-A2402 fused to the murine a3, transmembrane and cytoplasmic domains of H-2 K^{b}, can be produced using methods described above (see Gotoh et al., Int. J. Cancer 100:565 (2002)).

Any promoter capable of directing expression of HLA-A24 in at least the hematopoietic tissues of mice can be used, including, but are not limited to, cytomegalovirus (CMV) promoter, the SV40 early promoter region, the promoter contained in the 3′ long terminal repeat of Rous sarcoma virus, the herpes tk promoter, and the regulatory sequences of the metallothionein gene, as well as the HLA-A24 promoter itself. Preferred promoters are those promoters with organism-wide expression. Tissue specificity should come from the location of the tumors, which are the only cells expressing the new tumor-specific peptide antigen.

The double transgenic offspring can be used to identify antigen-specific cancer immunotherapies, such as peptide vaccines (including peptide modification), adjuvant administration, cytokine and co-stimulatory molecule administration, and negative regulator molecule blockade, that reduce, reverse or inhibit β-catenin-induced tumor formation (or some other phenotype associated with β-catenin expression) in the mice as compared to double transgenic mice not administered the immunotherapy regimen. The effectiveness of the immunotherapy regimen can be confirmed by effects on pathological changes or by induction of peptide specific CTL response when the animal dies or is sacrificed (see Miconnet et al., J. Immunol. 166:4612 (2001)). Such immunotherapies are candidates for development of treatments for cancers associated with β-catenin activation, such as colon cancer, brain cancer, gastric cancer, thyroid cancer, skin cancer, prostate cancer, kidney cancer, liver cancer, ovarian cancer, and uterine cancer.

In addition, the transgenic β-catenin mouse, the transgenic LEF/TCF-reporter mouse, and the transgenic HLA-A2402/K^{b} mouse can all be crossed to produce a triple transgenic mouse that provides a non-invasive, sensitive screening method for identifying immunotherapies without the need to sacrifice the animal. For example, the double transgenic β-catenin/LEF/TCF-reporter mouse can be mated with the transgenic HLA-A2402/K^{b} mouse and resulting offspring screened for the presence of all three transgenes.

Even though the description relates to mice, any animal may be used in the practice of the invention. However, the use of mice is preferred.

Specific embodiments according to the methods of the present invention will now be described in the following examples. The examples are illustrative only, and are not intended to limit the remainder of the disclosure in any way.

### EXAMPLES

### Example 1

### Generation of Transgenic β-Catenin Mouse

A variation of the “hit and run” gene targeting technique (Hasty et al., Nature 350:243 (1991)) was used to produce a mouse strain containing a latent oncogenic allele of β-catenin capable of spontaneous activation in vivo. The S37F allele of β-catenin, in which the serine at residue 37 in exon 3 is replaced by phenylalanine, was chosen because it removes a major GSK3β phosphorylation site and thus stabilizes β-catenin. Mutations in the S37 codon of β-catenin have been identified in a variety of human cancers, including colorectal, hepatocellular, ovarian, uterine and thyroid (Polakis, Genes & Dev. 14:1837 (2000)).

Construction of the Targeting Vector

A 5.6-kb EcoRI-NdeI fragment of the murine β-catenin gene containing exon 2 to 7 sequences was isolated and subcloned into pSP72 (Promega, Madison, Wis.). A 1.5-kb fragment of the neomycin resistance gene was inserted into the EcoRI-ClaI restriction sites. Site directed mutagenesis was performed using the GeneEditor™ In Vitro Site-Directed Mutagenesis System (Promega) according to manufacturer's instructions to create the pβ-catenin S37F allele. The sequence of the oligonucleotide used to create the EcoRI site and S37F mutation in exon 3 was 5′-TTA CTT GGA TTC TGG AAT TCA TTT CGG TGC CAC CAC CAC AGC-3′ (SEQ ID NO:1).

Construction of Probes

To generate an internal probe, a 256-bp fragment of exon 4 of the pβ-catenin gene was amplified using the forward and reverse primers 5′-ATA TTG ACG GGC AGT ATG CAA TG-3′ (SEQ ID NO:2) and 5′-CCT GGT CCT CAT CGT TTA GCA GTT-3′ (SEQ ID NO:3), respectively. For the external probe, a 280-bp NdeI-BglI fragment corresponding to the intron between exons 7 and 8 of the pβ-catenin gene was amplified using the forward and reverse primers 5′-CAT ATG GAG AAC ACT GCT TAA TG-3′ (SEQ ID NO:4) and 5′-CTC TTC TAA GAC ACT CCT CTT C-3′ (SEQ ID NO: 5), respectively. The two probes were labeled with 50 μCi of ³²p using the Ready-To-Go™ Labeling Kit (Amersham Biosciences, Piscataway, N.J.)

Targeting and Characterization of ES Cells

pβ-catenin S37F was linearized in intron 6 of the pβ-catenin gene and transfected into mouse 129/SV ES cells. Following selection with neomycin for 14 days, genomic DNA from neo resistant clones was digested with BglI, electrophoresed on 0.8% agarose gel and transferred onto Hybond membranes (Amersham Biosciences). The membranes were hybridized using the external intron 7 probe overnight at 68° C., then washed 3 times with 2×SSC, 0.5% SDS at room temperature, and 2 times in 0.1×SSC, 0.5% SDS at 68° C. The two expected 8631 and 6151-bp bands were observed in DNA from clones IIA6(2) and IIG3(3), indicating that they had integrated the transgene by homologous recombination.

In order to determine the configuration of the integrated gene in clones IIA6(2) and IIG3(3), DNA was digested with EcoRI and BglI and analyzed by Southern blot as described above using the internal exon 4 probe. Clones IIA6(2) and IIG3(3) presented the same profile with the two expected 5558 and 4520-bp bands, indicating an integration of the transgene 3′ of the wild-type exon 3. Thus, clones IIA6(2) and IIG3(3) were found to have a wild-type β-catenin allele on one chromosome and a latent, non-functional β-catenin allele made up of wild-type sequence and mutant S37F sequence separated by plasmid and neomycin-resistant sequences on the other chromosome.

Clones IIA6(2) and IIG3(3) were microinjected into FVB blastocyst-stage embryos and implanted into pseudo-pregnant females for development. The resulting chimeric animals were mated with wild-type C57BL/6 mice and F1 offspring screened by PCR for germ-line transmission. A 660-bp fragment of exon 3 was amplified with the forward and reverse primers 5′-TCA ACC CAC TTG TGC TGT G-3′ (SEQ ID NO:6) and 5′-CGT GGA CAA TGG CTA CTC AAG-3′ (SEQ ID NO:7), respectively. The amplimer generated was then subjected to digestion by EcoRI and agarose gel electrophoresis. S37F mice, which contain a novel EcoRI site associated with the S37F mutation, generated two fragments of 405 and 255-bp. Approximately 50% of all tested mice contained the S37F mutation.

Characterization of Transgenic β-Catenin Mice

It was expected that the dividing cells of the transgenic β-catenin mice will resolve the duplication in the latent oncogenic β-catenin allele by an intrachromosomal or sister chromatid recombination event at a low frequency in vivo, resulting in activation of the oncogenic allele. This spontaneous activation was confirmed by histologic examination of F1 mice, which revealed localized peri-mammary skin papillomas at 4 months and intestinal-type gastric metaplasia and dysplasia at 8-10 months. Gastric lesions were found histologically to be multifocal and sporadic, thereby exhibiting a greater similarity to human cancer than previous transgenic β-catenin mice.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

We claim:

1. A transgenic β-catenin mouse whose genome comprises a latent oncogenic β-catenin allele capable of spontaneous activation in vivo.

2. A transgenic β-catenin mouse comprising at least one cell expressing an oncogenic form of β-catenin, wherein the genome of the transgenic mouse comprises a latent oncogenic β-catenin transgene which upon spontaneous activation in vivo results in at least one cell of said transgenic mouse expressing an oncogenic form of β-catenin, and wherein expression of said oncogenic form of β-catenin results in said transgenic mouse developing a clonally derived sporadic tumor.

3. The transgenic β-catenin mouse of claim 1 or 2, wherein the oncogenic form of β-catenin contains an amino-terminal serine or threonine modification or mutation, or an amino acid adjacent to them, such that post-translational phosphorylation and subsequent degradation of β-catenin is inhibited.

4. The transgenic β-catenin mouse of claim 3, wherein the oncogenic form of β-catenin is S37F.

5. A method of producing the transgenic β-catenin mouse of claim 1 comprising: (a) providing a transgene comprising an oncogenic β-catenin nucleotide sequence; (b) introducing said transgene into mouse embryonic stem cells; (c) selecting embryonic stem cells that have integrated said transgene by homologous recombination such that a latent oncogenic β-catenin allele is formed; (d) introducing said embryonic stem cells containing said transgene into mouse blastocysts; (e) transplanting said blastocytes into a pseudopregnant mouse; (f) allowing said embryo to develop to term, producing a chimeric transgenic mouse; and (g) breeding chimeric transgenic mice to obtain F1 mice heterozygous for the transgene.

6. A method for identifying a compound for treating a cancer associated with β-catenin activation comprising administering to a first β-catenin transgenic mouse of claim 1 or 2 a candidate agent and determining the beneficial effect of the candidate agent upon the first transgenic mouse as compared to a second β-catenin transgenic mouse of claim 1 or 2 not administered the agent.

7. The method of claim 6, wherein the beneficial effect is reduced activation of LEF/TCF target genes.

8. The method of claim 6, wherein the beneficial effect is delayed death or prevention, reduction, or regression of tumor formation.

9. The method of claim 8, wherein the beneficial effect is confirmed by pathological examination.

10. The method of claim 6, wherein the candidate agent is administered prior to spontaneous activation of the latent oncogenic β-catenin allele.

11. The method of claim 6, wherein the candidate agent is administered following spontaneous activation of the latent oncogenic β-catenin allele.

12. The method of claim 6, wherein the candidate agent is administered prior to tumor development.

13. The method of claim 6, wherein the candidate agent is administered following tumor development.

14. A double transgenic mouse whose genome comprises a latent oncogenic β-catenin allele capable of spontaneous activation in vivo and a reporter transgene operably linked to a β-catenin-inducible promoter.

15. A double transgenic mouse comprising at least one cell expressing an oncogenic form of β-catenin, wherein the genome of the transgenic mouse comprises a reporter transgene operably linked to a β-catenin-inducible promoter and a latent oncogenic β-catenin transgene which upon spontaneous activation in vivo results in at least one cell of said double transgenic mouse expressing an oncogenic form of β-catenin, and wherein expression of said oncogenic form of β-catenin induces expression of said reporter transgene in said double transgenic mouse.

16. The double transgenic mouse of claim 14 or 15, wherein the oncogenic allele of β-catenin alters an amino-terminal serine or threonine residue of β-catenin, or an amino acid adjacent to them, such that post-translational phosphorylation and subsequent degradation of β-catenin is inhibited.

17. The double transgenic mouse of claim 16, wherein the oncogenic allele of β-catenin is S37F.

18. The double transgenic mouse of claim 14 or 15, wherein the reporter transgene is luciferase.

19. The double transgenic mouse of claim 14 or 15, wherein the β-catenin-inducible promoter is LEF/TCF.

20. A method of producing the double transgenic mouse of claim 14 comprising crossing the transgenic β-catenin mouse of claim 1 with a transgenic mouse whose genome comprises a reporter transgene operably linked to a β-catenin-inducible promoter, and screening the resulting offspring for the presence of the latent oncogenic β-catenin allele and the reporter transgene.

21. A method for identifying a compound for treating a cancer associated with β-catenin activation comprising administering to a first double transgenic mouse of claim 14 or 15 a candidate agent and determining the effect of the candidate agent upon reporter transgene expression in said first double transgenic mouse as compared to a second double transgenic mouse of claim 14 or 15 not administered the agent.

22. The method of claim 21, wherein the reporter transgene is luciferase.

23. The method of claim 21, wherein the candidate agent is administered prior to reporter transgene expression.

24. The method of claim 21, wherein the candidate agent is administered following reporter transgene expression.

25. The method of claim 22, wherein the determining step comprises monitoring luciferase expression for reduced bioluminescence using a charge-coupled device (CCD) camera following administration of a luciferin substrate to the double transgenic mouse.

26. A double transgenic mouse whose genome comprises a latent oncogenic β-catenin allele capable of spontaneous activation in vivo and a HLA-A24 transgene.

27. A double transgenic mouse comprising at least one cell expressing an oncogenic form of β-catenin, wherein the genome of the transgenic mouse comprises a HLA-A24 transgene and a latent oncogenic β-catenin transgene which upon spontaneous activation in vivo results in at least one cell of said transgenic mouse expressing an oncogenic form of β-catenin and HLA-A24, and wherein expression of said oncogenic form of β-catenin results in said double transgenic mouse developing a clonally derived sporadic tumor.

28. The double transgenic mouse of claim 26 or 27, wherein the oncogenic allele of β-catenin alters an amino-terminal serine or threonine residue of β-catenin, or an amino acid adjacent to them, such that post-translational phosphorylation and subsequent degradation of β-catenin is inhibited.

29. The double transgenic mouse of claim 28, wherein the oncogenic allele of β-catenin is S37F.

30. The double transgenic mouse of claim 26 or 27, wherein the HLA-A24 transgene is HLA-A2402/Kb.

31. A method of producing the double transgenic mouse of claim 26 comprising comprising crossing the transgenic β-catenin mouse of claim 1 with a transgenic mouse whose genome comprises an HLA-A24 transgene and screening the resulting offspring for the presence of the latent oncogenic β-catenin allele and the HLA-A24 transgene.

32. A method for identifying an antigen-specific cancer immunotherapy for treating a cancer associated with β-catenin activation comprising administering to a first double transgenic mouse of claim 26 or 27 an immunotherapy regimen and determining the beneficial effect of the immunotherapy regimen upon said first double transgenic mouse as compared to a second double transgenic mouse of claim 26 or 27 not administered the immunotherapy regimen.

33. The method of claim 32, wherein the beneficial effect is reduced activation of LEF/TCF target genes.

34. The method of claim 32, wherein the beneficial effect is delayed death or prevention, reduction, or regression of tumor formation.

35. The method of claim 33, wherein the beneficial effect is confirmed by pathological examination.

36. The method of claim 33, wherein the beneficial effect is confirmed by induction of a peptide specific CTL response.

37. The method of claim 32, wherein the immunotherapy regimen is administered prior to spontaneous activation of the latent oncogenic β-catenin allele.

38. The method of claim 32, wherein the immunotherapy regimen is administered following spontaneous activation of the latent oncogenic β-catenin allele.

39. The method of claim 32, wherein the immunotherapy regimen is administered prior to tumor development.

40. The method of claim 32, wherein the immunotherapy regimen is administered following tumor development.

41. The method of claim 32, wherein the immunotherapy regimen is selected from the group consisting of peptide vaccination, adjuvant administration, cytokine administration, co-stimulatory molecule administration, negative regulator molecule blockade, and combinations thereof.
